(19) United States
(12) Patent Application Publication
(10) Pub. No.: US 2009/0010888 A1
(43) Pub. Date: Jan. 8, 2009

(54) Use of cytochrome P450 reductase as insecticidal target

(75) Inventors: Mark John Ingraham Paine, Dundee (GB); Charles Roland Wolf, Inchture (GB); Lesley Ann McLauchlin, Dundee (GB)

Correspondence Address: NIXON & VANDERHYE, PC, 901 NORTH GLEBE ROAD, 11TH FLOOR, ARLINGTON, VA, 22203, US

(73) Assignees: University Court of the University of Dundee, Dundee (GB); The University of Liverpool, Liverpool (GB)

(21) Appl. No.: 11/812,989

(22) Filed: Jun. 22, 2007

Related U.S. Application Data
(63) Continuation of application No. PCT/GB05/05040, filed on Dec. 23, 2005.

(30) Foreign Application Priority Data
Dec. 23, 2004 (GB) 0428186.1

(51) Int. Cl. A61K 35/76; A61K 31/7088; C12Q 1/68; G06G 7/50; G01N 33/53

(52) U.S. Cl. 424/93.2; 514/44; 435/6; 703/11; 435/7.1

(57) ABSTRACT

The invention provides a method of enhancing the effectiveness of pesticides, as well as pesticidal formulations. Furthermore, it also provides the means for the de novo rational design of pesticides. The present invention also relates to a method of screening agents for potential use in insecticides, particularly against mosquitoes.

### FIELD OF THE INVENTION

The invention provides a method of enhancing the effectiveness of pesticides, as well as pesticidal formulations. Furthermore, it also provides the means for the de novo rational design of pesticides. The present invention also relates to a method of screening agents for potential use in insecticides, particularly against mosquitoes.

### BACKGROUND OF THE INVENTION

The vast majority of pesticides are chemical agents. As has been widely recognized, the use of chemical pesticides has a number of disadvantages. Conventional chemical insecticides frequently act non-specifically, killing beneficial insect species in addition to the intended target. Chemicals may persist in the environment and present a danger to organisms higher up in the food chain than the insect pest. Exposure to chemical pesticides is hazardous and poses a threat to local animals and humans. In addition, resistant pest populations frequently emerge with repeated applications of pesticides. There is thus a need to develop means which will enable lower amounts and/or more specific pesticides to be used.

It is amongst the objects of the present invention to obviate and/or mitigate at least one of the aforementioned disadvantages.

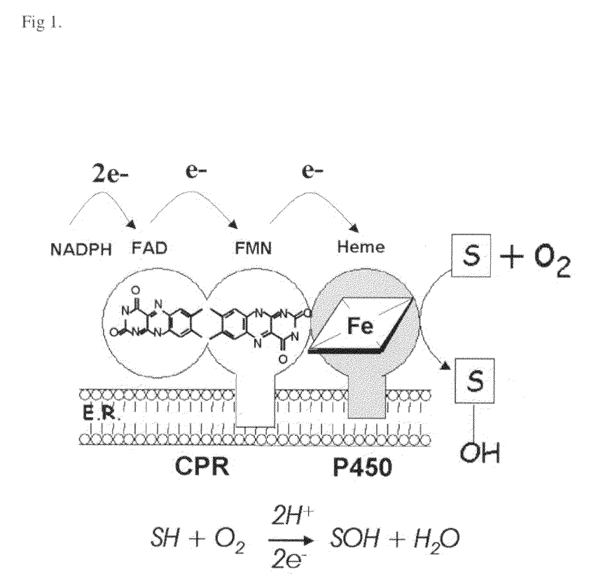

Previous genetic and biochemical evidence suggest the involvement of the cytochrome P450 gene family in resistance mechanisms through insecticide metabolism^{1,2}. In the A. gambiae genome at least 111 P450 genes have been annotated but only a single gene for the obligate P450 redox partner NADPH cytochrome P450 oxidoreductase (CPR)¹. The cytochrome P450 gene family is linked with a host of critical biochemical pathways including insecticide metabolism and the development of insecticide resistance². Defining precise roles for P450 gene family members is difficult due to the large numbers of P450 genes and their overlapping substrate specificities. However, the mono-oxygenation reaction performed by P450 requires electrons which are solely supplied by cytochrome P450 reductase (CPR), a diflavin enzyme that contains FMN and FAD cofactors, which transfers electrons from the reduced form of nicotinamide adenine dinucleotide phosphate (NADPH) through a series of redox-coupled reactions to P450³ (FIG. 1). Thus inhibition of CPR will effectively shut down all microsomal P450 activity, adversely affecting key physiological functions, including their chemoprotective roles⁴. Although not its prime function, CPR may also couple with other redox partners such as heme oxygenase³, adding to the debilitating effects of CPR shutdown.

We note here also that P450s are generally divided into two major classes (Class I and Class II) according to the different types of electron transfer systems they use³. P450s in the Class I family include mitochondrial P450s, which use a two-component shuttle system consisting of an iron-sulfur protein (ferredoxin) and ferredoxin reductase. The Class II enzymes are the microsomal P450s, which receive electrons from a single membrane bound enzyme, NADPH cytochrome P450 reductase (CPR), which contains FAD and FMN cofactors (FIG. 1). Cytochrome b₅ may also couple with some members of the class II 450s family, including insects, to enhance the rate of catalysis². Multiple P450s capable of metabolising xenobiotic (foreign) compounds have been linked to P450s that are expressed in the mitochondria². As for the Type II system that uses a single CPR gene, the whole range of mitochondrial P450s are furnished with electrons by a single ferredoxin/ferredoxin reductase couplet. Thus inactivation of the ferredoxin/ferredoxin reductase system will effectively shut down all mitochondrial P450 activity, adversely affecting key physiological functions, including their chemoprotective roles as well.

The present invention is based in part on the critical role of CPR in the P450 monooxygenase complex in insects/pests and how this may be utilised to screen for novel insecticides/pesticides and/or enhancing existing insecticide/pesticide treatment.

### SUMMARY OF THE INVENTION

In a first aspect there is provided a method of pest treatment comprising:

an effective amount of an agent to a pest in order to reduce cytochrome P450 reductase (CPR) expression and/or functional activity in said pest, wherein the agent is capable of reducing CPR expression and/or activity.

Typically, a pesticide may be administered, concurrently, or otherwise along with the agent.

CPR expression may be reduced, for example, by use of antisense oligonucleotides designed against the CPR gene and/or promoter sequences, or by RNAi techniques known in the art. For RNAi, typically a double stranded RNA (dsRNA) fragment of 100 bp-1 kb, e.g. 300 bp-700 bp in length may be generated for administering to the pest. The dsRNA may be generated so as to be capable of inhibiting/reducing expression of the CPR gene within the pest⁵⁻⁸.

Chemical agents such as dipheyliodonium (DPI) or other known inhibitors of flavin containing enzymes may be used to reduce CPR activity. These include iodonium compounds iodonium diphenyl, di-2-thienyliodonium, phenoxiaiodonium as well as NADP, fragments of NADP and nucleotide analogues such as NAD, 2′-AMP and 2′-5′-ADP.

The present inventors have observed that, for example, a reduction in the expression or activity of CPR in the mosquito results in an increase in the efficacy of an existing pesticide, permethrin, when administered to mosquitoes.

In a further aspect, there is provided a method of killing insects, especially mosquitoes comprising administering a pyrethroid insecticide, such as permethrin, in combination with a double-stranded RNA molecule corresponding to at least a portion of the gene sequence of the insect's CPR gene, for inhibiting expression of the mosquito's CPR gene, by the process of RNAi or a CPR inhibitor such as DPI, for reducing CPR activity.

In a yet further aspect there is provided a pesticide formulation for use in killing pests, the formulation comprising a pesticidal agent, e.g. a pyrethroid, such as permethrin and a dsRNA molecule corresponding to at least a portion of the gene sequence of the pest's CPR gene, or a CPR inhibitor, such as DPI.

Typical pests which are targets of the present invention include Dictyoptera (cockroaches); Isoptera (termites); Orthoptera (locusts, grasshoppers and crickets); Diptera (house flies, mosquito, tsetse fly, crane-flies and fruit flies); Hymenoptera (ants, wasps, bees, saw-flies, ichneumon flies and gall-wasps); Anoplura (biting and sucking lice); Siphonaptera (fleas); and Hemiptera (bugs and aphids), as well as arachnids such as Acari (ticks and mites) and insect bourne protozoan parasites (Trypanosoma, Leishmania, Giardia, Trichomonas, Entamoeba, Naegleria, Acanthamoeba, Plasmodium, Toxoplasma, Cryptosporidium, Isospora and Balantium)

It is to be understood that the term “corresponding” is taken to mean that the double-stranded RNA molecule is capable of specifically hybridising to mRNA encoding the CPR protein from the pest. The dsRNA as used herein, refers to a polyribonucleotide structure which is formed by either a single self-complementary RNA strand or by at least two complementary RNA strands. The degree of complimentarily need not be 100%. Rather, it must be sufficient to allow the formation of a double-stranded structure under the conditions employed.

In a yet further aspect there is provided a pesticide formulation, the formulation comprising a pesticidal agent, e.g. a pyrethroid, such as permethrin, and a genetically engineered insect virus which comprises an inserted nucleic acid encoding at least a portion of a pest's CPR gene and wherein said nucleic acid is capable of being expressed as a dsRNA molecule.

Pests are treated (or their loci treated) with a combination of dsRNA or recombinant virus capable of expressing a dsRNA molecule designed to inhibit CPR expression in the pest, by the process of RNA inhibition and a pesticide. The recombinant virus preferably is a baculovirus that expresses said dsRNA molecule in pest cells infected with the recombinant baculovirus.

Treatments in accordance with the invention can be simultaneous (such as by applying a pre-mixed composition of dsRNA/recombinant virus/CPR inhibitor and pesticide). Alternatively, the pests or loci may first be treated by applying the dsRNA recombinant virus/CPR inhibitor followed by pesticide within about 24 hours.

The present invention encompasses the use of genetically engineered insect viruses in combination with chemical insecticides to treat pests such as insects, especially mosquitoes. Although baculoviruses are specifically mentioned, as an illustration, this invention can be practiced with a variety of insect viruses, including DNA and RNA viruses.

By “baculovirus” is meant any baculovirus of the family Baculoviridae, such as a nuclear polyhedrosis virus (NPV). Baculoviruses are a large group of evolutionarily related viruses, which infect only arthropods; indeed, some baculoviruses only infect insects that are pests of commercially important agricultural and forestry crops, while others are known that specifically infect other insect pests. Since baculoviruses infect only arthropods, they pose little or no risk to humans or the environment.

Suitable baculoviruses for practicing this invention may be occluded or non-occluded. The nuclear polyhedrosis viruses (“NPV”) are one baculovirus subgroup, which are “occluded”. That is, a characteristic feature of the NPV group is that many virions are embedded in a crystalline protein matrix referred to as an “occlusion body”. Examples of NPVs include Lymantria dispar NPV (gypsy moth NPV), Autographa californica MNPV, Anagrapha falcifera NPV (celery looper NPV), Spodoptera litturalis NPV, Spodoptera frugiperda NPV, Heliothis armigera NPV, Mamestra brassicae NPV, Choristoneura fumiferana NPV, Trichoplusia ni NPV, Heliocoverpa zea NPV, and Rachiplusia ou NPV. For field use occluded viruses are preferable due to their greater stability since the viral polyhedrin coat provides protection for the enclosed infectious nucleocapsids. Particularly preferred viruses which may be used to infect mosquitoes include:

1. CuniNPV (family Baculoviridae, genus Nucleopolyhedrovirus) found in field populations of the mosquitoes C. nigripalpus and C. quinquefasciatus (vectors of St Louis and Eastern equine encephalitis)^{9,10}

2. UrsaNPV, a nucleopolyhedrovirus from the mosquito Uranotaenia sapphirina¹¹.

3. Recombinant Sindbis viruses have also been used alongside RNA interference (RNAi) as a potential anti-viral, intracellular pathway in the mosquito species Aedes aegypti (the vector for Dengue viruses (i) DENV)) to reduce vector competence to DENV¹². These viruses have been used to trigger expression of DENV-derived RNA segments that when expressed in mosquitoes ablate homologous DENV replication and transmission; and

4. Semliki Forest virus (SFV) expressing T7 RNA polymerase (T7-RP), has been shown to drive transient expression of the chloramphenicol acetyltransferase (cat) gene in mammalian and mosquito cells after transfection of plasmids carrying the reporter gene under the control of the T7 promoter¹³.

Further suitable baculovirus vectors suitable for use in the present invention are described, for example, in U.S. Pat. No. 6,326,193, to which the reader is directed and the incorporation of which is included herein by reference thereto.

The pesticides with which the present method may be practiced include: Na.sup+channel agonists (i.e. pyrethroids), Na.sup+channel blocking agents (i.e. pyrazolines), acetylcholinesterase inhibitors (i.e. organophosphates and carbamates), nicotinic acetylcholine binding agents (e.g. imidacloprid), gabaergic binding agents (e.g. emamectin and fipronil), octapamine agonists or antagonists (i.e. formamidines), and oxphos uncouplers (e.g. pyrrole insecticides).

As will be exemplified further in the examples section, the present inventors have found that by reducing the expression of pests CPR in at least a selection of cells within the pest, results in the pest being more susceptible to a pesticide. By more susceptible is meant that less pesticide and/or a shorter period of administration is required to kill pests in comparison to using the same pesticide without concurrent reduction in expression of the pest's CPR. Conveniently, less than 90%, 80%, 70%, 60%, 50%, 40%, 30%, 20% or 10% of the amount of pesticide required to kill the pest in the absence of the reduction in expression or activity of CPR is required.

It should be noted that it is not generally necessary to reduce CPR expression in all the cells of the pest which express CPR. Conveniently, at least a reduction in CPR expression may be observed in gut cells, in particular oenocytes. Inhibition of CPR expression may easily be quantified by, for example, either the endogenous CPR RNA or CPR protein produced by translation of the CPR RNA and such techniques are readily known to the skilled addressee, see for example Sambrook et al.¹⁴

As is well known, pesticides may be applied by means such as spraying, atomising, dusting, scattering or pouring and may be formulated for such applications as powders, dusts, granulates, as well as encapsulations such as in polymer substances. When practicing this invention such conventional application means may be used. Preferably, the pesticide and for example dsRNA/recombinant baculovirus may be admixed in desired proportions, and may typically include inert carriers such as clay, lactose, defatted soy bean powder, and the like to assist in application.

However, it is possible to apply compositions including each component separately, by utilizing the dsRNA/baculovirus or CPR inhibitor first then followed (preferably within about forty-eight hours) by the pesticide. When the baculovirus/CPR inhibitor is first used (followed by pesticide), then the baculovirus/inhibitor can be applied by conventional means, such as spraying.

In a further aspect there is provided a method of screening for a potential pesticide comprising the steps of:

a) providing a pest cytochrome P450 reductase (CPR) model system comprising CPR from a pest organism and a substrate capable of being reduced by said CPR;

b) contacting a test pesticide agent with said system;

c) initiating reduction of said substrate by the addition of an electron donor to said system; and

d) observing any change in a rate of substrate reduction in comparison to a rate of substrate reduction in the absence of said test insecticide agent.

The above aspect is based on observations by the present inventors that the CPR from the mosquito, Anopheles gambiae, is biochemically different to human CPR in the binding for adenosine comprising molecules and also more sensitive to certain drug agents. Without wishing to be bound by theory, it is thought that the mosquito CPR may be structurally different to CPRs from other species, leading to differences in response to various chemical agents.

NADPH cytochome P450 reductase (CPR) (EC1.6.2.4) is a microsomal dual flavin redox protein. Its main function is the transfer of electrons from NADPH via FAD and FMN cofactors to cytochrome P450 isoenzymes (see FIG. 1). The CPR model systems of the present invention generally comprise CPR and a substrate which is capable of being reduced by said CPR. Essentially, any suitable reduction system may be employed, as long as it is possible to detect, in some manner, the reduction of the substrate by action of the CPR. Such a system can be as shown in FIG. 1, or modified versions thereof which utilise a fluorescent compound such as 7-ethoxyresorufin-O-dealkylase (EROD), which is converted to resorufin following oxidation of a cytochrome P450 enzyme. Alternatively simpler systems which employ an alternative substrate to cytochrome P450 may be employed. A list of commonly used substrates and methods for measuring CPR function includes:
1. cytochrome c, a facile electron acceptor reduced by CPR and related diflavin enzymes such as nitric oxide synthase; reduction of cytochrome c can be followed spectrometrically at 550 nm¹⁵.
2. Artificial electron accepting compounds¹⁵ such as potassium ferricyanide (A₄₂₀ nm), 2,6-dichloroindophenol (DCIP) (A₆₀₀ nm), 3-acetylpyridine adenine dinucleotide phosphate (A₃₆₃ nm), menadione (reduction determined indirectly by following NADPH oxidation).
3. NADPH oxidation, which can be followed at 340 nm¹⁵.

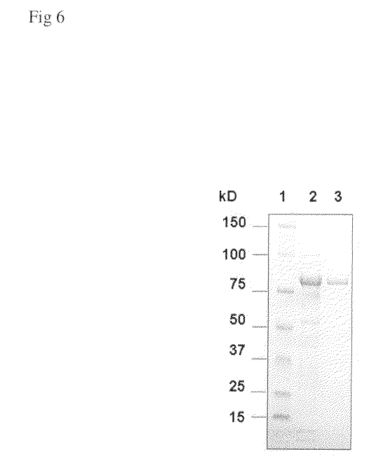

The preferred pest CPR is the mosquito CPR. The pest CPR may be provided in a purified form as shown FIG. 6 (i.e. substantially isolated from other proteins), or alternatively cells which express significant levels of CPR may be isolated and used in said method. Suitable cells may be commonly used cells which are capable of expressing foreign recombinant vehicles such as E. coli¹⁶, yeast, and Spodopteran cells infected by baculovirus¹⁷. Where crude cell fractions expressing recombinant CPR may be used to monitor the enzyme's function, endogenous insect cells for example, the oenocytes, antenna and/or midgut epithelia from A. gambiae may be used. Most preferably the cells are oenocytes, which appear to express CPR at high levels.

Purified CPR may be obtained by cloning and expression of the CPR cDNA as known in the art and/or as described hereinafter.

The test pesticide agent may be any suitable molecule, such as a small novel or known organic molecule. The provision of candidate molecules for use in the present invention are well known to those skilled in the art. For example libraries of compounds can be easily synthesised and tested. This is well described for example in: Applications of combinatorial technologies to drug discovery, 2. Combinatorial organic synthesis, library screening techniques, and future direction, J. Med. Chem., 1994, 37, 1385-1401. Alternatively existing chemical molecule libraries may be tested. The test insecticide may also be an analogue of a known insecticide or may be a nucleoside analogue designed to disrupt binding of NADPH to said CPR.

Typically the electron donor is NADPH, and may be obtained readily from commercial sources e.g. Sigma-Aldrich.

In order to be able to ascertain whether or not said test pesticide molecule is having any effect on said CPR system, it is necessary to compare the rate of reduction of said substrate in the absence of said test pesticide. In this manner, it is possible to determine whether or not the test pesticide displays little or no effect on the CPR system, or causes an increase or decrease in the rate of substrate reduction. It is envisaged that test pesticides which cause a reduction of substrate reduction may be of most potential utility, but compounds which increase reduction of the substrate may also find application. A suitable pesticide molecule may be a compound that strongly modulates, either agonistically or antagonistically, the activity of CPR. Thus, the purpose of the test methods described herein, may be the selection of pesticides, that may interfere with CPR to modulate activity of the protein and/or RNA or protein expression levels.

Once a molecule has been identified as having an effect on CPR activity in vitro, further testing may be carried out on live organisms. Thus, in a further step, each potentially useful pesticide may then be tested directly for killing activity on pests, especially insects. For example, in a typical fly killing assay, young flies are kept without fluid for a time, then transferred to vials containing filter paper dosed with a solution of the chemical to be tested. A range of chemical concentrations (e.g. 10⁻²-10⁻¹⁰M) may be used. After a defined treatment, flies are returned to normal conditions and observed. Rate of killing and percentage lethality are the parameters assessed.

It may also be desirable to test said molecules on a similar human or mammalian model system, so that it may be possible to select molecules which do not display a significant deleterious effect on human or mammalian CPR/cytochrome P450 systems.

In an another aspect, the present invention provides systems, particularly a computer system, intended to generate structures and/or perform rational drug design for Anapheles sp., especially Anopheles gambiae P450 reductase, or homologues or mutants, the system containing either (a) atomic coordinate data, said data defining the three-dimensional structure of said Anopheles P450 reductase, or at least selected coordinates thereof; (b) structure factor data of said Anopheles P450 reductase recorded thereon, the structure factor data being derivable from the atomic coordinate data or (c) a Fourier transform of atomic coordinate data or at least selected coordinates thereof.

The skilled addressee will readily understand how to obtain such data by expressing the Anopheles P450 reductase in order to obtain purified Anopheles P450 reductase and thereafter crystallising said Anopheles P450 reductase and subjecting said crystal(s) to x-ray crystallographic techniques known in the art in order to obtain atomic coordinates; for example as performed with rat CPR¹⁸ (PDB accession number: 1AMO).

Such data is useful for a number of purposes, including the generation of structures to analyse the mechanisms of action of said P450 reductase, and/or to perform rational insecticide design of compounds which interact with said reductase, such as modulators of reductase activity, e.g. activators or inhibitors.

In a further aspect, the present invention provides computer readable media with either (a) atomic coordinate data recorded thereon, said data defining the three-dimensional structure of Anopheles sp., especially Anopheles gambiae P450 reductase, or at least selected coordinates thereof, (b) structure factor data for said Anopheles P450 reductase recorded thereon, the structure factor data being derivable from the atomic coordinate data or (c) a Fourier transform of said atomic coordinate data, or at least selected coordinates thereof.

By providing such computer readable media, the atomic coordinate data can be routinely accessed to model Anopheles sp. P450 reductase or selected coordinates thereof. For example, RASMOL (Sayle et al., TIBS, Vol. 20, (1995), 374) is a publicly available computer software package which allows access and analysis of atomic coordinate data for structure determination and/or rational drug design.

On the other hand, structure factor data, which are derivable from atomic coordinate data (see e.g. Blundell et al., in Protein Crystallography, Academic Press, New York, London and San Francisco, (1976)), are particularly useful for calculating e.g. difference Fourier electron density maps.

In another aspect, the present invention provides methods for modelling the interactions between Anopheles sp. such as Anopheles gambiae P450 reductase and modulators of said reductase activity. Thus there is provided a method for modelling the interaction between Anopheles sp. P450 reductase and an agent compound which modulates said reductase activity, comprising the steps of:

(a) employing three-dimensional atomic coordinate data to characterise the Anopheles sp. P450 reductase binding site; (b) providing the structure of said agent compound; and (c) fitting said agent compound to the binding site.

The present invention further provides a method for identifying an agent compound (e.g. an inhibitor) which modulates Anopheles sp. P450 reductase activity, comprising the steps:

(a) employing three-dimensional atomic coordinate data to characterise at least one Anopheles sp. P450 reductase binding site; (b) providing the structure of a candidate agent compound; (c) fitting the candidate agent compound to the binding site(s); and (d) selecting the candidate agent compound.

Thus the present invention enables the design of inhibitors which may be specific for only the Anopheles sp. P450 reductase. That is to say, the candidate agent compound may be a better fit to the Anopheles sp. P450 reductase binding site than to a corresponding binding site defined by the corresponding residues of another P450 reductase. Thus the method may involve the step of comparing the binding of the candidate agent compound to the Anopheles sp. P450 reductase binding site, and to a corresponding binding site defined by the corresponding residues of another P450 reductase.

The present inventors have observed that the Anopheles gambiae CPR does not substantially bind to ADP sepharose, whereas CPRs from other organisms have done so in the past. Without wishing to be bound by theory, it is thought that this is likely to be due to a difference in the NADPH binding domain of the A. gambiae CPR. This observation allows the possibility of screening for other organisms/pests which may also possess CPRs with altered NADPH binding domains, or at least NADPH binding domains which do not substantially bind ADP sepharose. This allows the identification of pests which are most likely to be susceptible to pesticides which have been identified/designed to inhibit CPRs from, for example, pests such as A. gambiae which possess unusual NADPH binding domains. Pesticides which target altered CPR NADPH binding domains, such as the CPR from A. gambiae, are likely to be highly specific and moreover, environmentally friendly, as most other organisms do not possess such altered NADPH binding domains.

Thus, in a further aspect there is provided a method of determining whether or not a pest is likely to be susceptible to a pesticide identified according to the present invention, comprising the steps of:

a) obtaining said pest;

b) homogenising said pest, so as to release said pest's cytochrome P450 reductase (CPR);

c) admixing said homogenate containing CPR with ADP sepharose—if necessary removing ADP sepharose binding contaminants from the homogenate first before the ADP-affinity binding step; and

d) detecting whether or not said CPR substantially binds to ADP sepharose.

Such a method may also be of use in detecting whether or not a particular pesticide is likely to continue to be of use in treating a particular pest. Given the propensity for pests to develop resistance through natural selection and alteration of enzyme structure/function, the present invention provides a means to monitor for pests which comprise non-2′-5′ ADP binding CPRs to see if a change to 2′-5′ ADP binding may occur over time.

Typically the pest may be homogenised, simply by grinding in a pestle and mortar, or using a homogeniser, known to the skilled addressee. In order to determine whether or not a pests CPR binds to ADP sepharose and therefore whether or not the CPR is a “conventional” or unusual CPR, the pest extract comprising CPR, may be added to a column comprising ADP sepharose and any CPR allowed to bind thereto. Unbound CPR will simply flow through the column and can be collected. Likewise, a ‘batch’ method may be employed whereby ADP-resin is simply added to the insect homogenate, mixed to allow adsorption, and centrifuged to pellet the ADP sepharose. Unbound CPR will remain in the supernatent, bound CPR will bind to the ADP sepharose). Some organisms contain a level of ADP-binding molecules that can compete and reduce the affinity for CPR. Therefore a clean-up step such as ion-exchange may be incorporated to remove such contaminants. In order to detect whether or not CPR is present bound to the ADP sepharose or in the unbound fraction, CPR activity may be detected as previously described, or CPR protein may be detected, for example, by way of an immunoassay using an antibody specifically reactive with said CPR, using techniques, such as western blotting, well known to those skilled in the art.

### DETAILED DESCRIPTION

The present invention will now be further described by way of example and with reference to the figures which show:

FIG. 1 shows a schematic view of the P450 mono-oxygenase complex; P450 catalyses the insertion of a single oxygen molecule into an organic substrate (S) to produce a mono-oxygenation product (S—OH) and water. Two electrons are supplied by NADPH and shuttled consecutively to the heme centre of P450 via the isoalloxazine rings of FAD and FAWN. The complex is tethered to the endoplasmic reticulum. The reaction scheme is shown at the bottom.

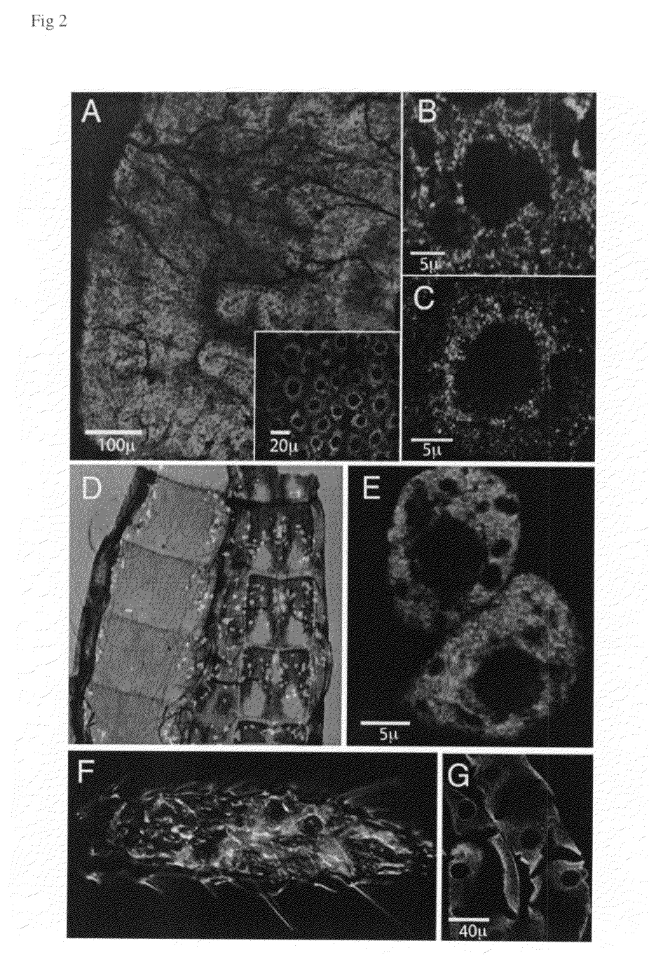

FIG. 2 shows immunolocalisation of CPR. CPR is labelled in green in all images. A, B, C—Midgut, A inset and C counterstained red with nuclear pore antiserum, B counterstained red with integrin antiserum. CPR is abundantly localised in the perinuclear region D, merged brightfield fluorescence image of abdomen wall showing intense staining of oenocytes. E, oenocytes—nuclei counterstained blue with TO-PRO3. CPR appears dispersed throughout the cell. Cells contain large vesicle structures F female antennae—counterstained blue with DAPI. CPR is localised to a subset of cells. G Malpighian tubules—counterstained red with nuclear pore antiserum. CPR is localised specifically to the large principal (type I) cells of the tubules.

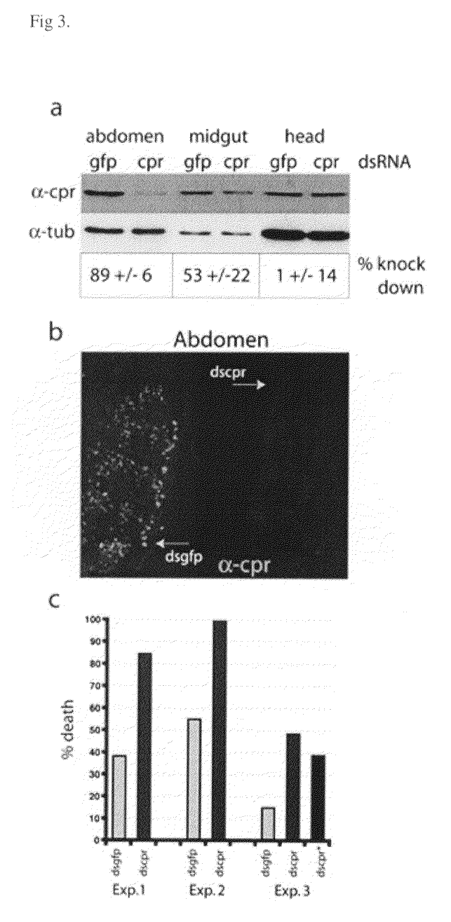

FIG. 3 shows silencing of CPR expression, a, immunoblot of total protein extracts from dissected body parts taken from mosquitoes injected with dsCPR (cpr) and dsGFP (gfp). Filters were probed with cpr antisera (α-cpr), then stripped and probed with tubulin antiserum (α-tub). The percentage of CPR remaining after knockdown was estimated by densitometric scanning of western filters using ImageJ software and corrected for loading using tubulin. The figures indicated are mean and SD from three independent experiments. Image shows random selected whole mount abdomens stains taken from control (con) or dscpr injected mosquitoes. Oenocyte staining is drastically reduced. b, Effect of CPR RNAi knockout on susceptibility to permethrin. Each independent experiment shows mean percentage numbers of mosquitoes dead, 24 hrs after permethrin treatment. Numbers refer to experiment number and * indicates the use of a different region of CPR to make dsRNA.

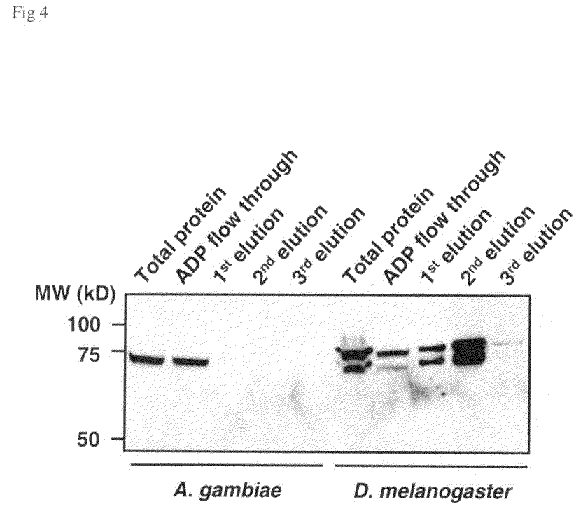

FIG. 4 demonstrates the different binding behaviours of CPR extracted from the mosquito A. gambiae and the closely related dipteran species, Drosophila melanogaster (fruit fly) with respect to 2′-5′-ADP. In this example, whole flies have been solubilized with a detergent/buffer solution, loaded onto 2′-5′-ADP sepharose mini-columns, washed and column bound proteins eluted with 50 mM 2′-AMP. (2′-5′-ADP Sepharose interacts strongly with NADP+-dependent dehydrogenases and other enzymes which have affinity for NADP+ (Amersham-Pharmacia Biotech 1999 handbook on Affinity Chromatography: Principles and Methods, edition AB). Thus a mixture of different NADP+ binding proteins will be eluted. To identify CPR, samples of whole fly extracts from the pre-2′-5′-ADP adsorption, post-2′-5′-ADP adsorption and post 2′-5′-ADP elution stages were transferred onto nitrocellulose by immunoblotting (Western Blotting) and CPR identified using rabbit antisera to A. gambiae CPR. In the An. gambiae lanes (FIG. 4), a prominant CPR band (˜75 kDa) is evident in the total protein and unbound (flow-through) lanes, but not in the eluted fraction. By contrast, in the D. melanogaster lanes, CPR bands are evident in total protein, the unbound fraction and elution fraction. Thus, the mosquito CPR is clearly different to the fruit fly with respect to the binding of 2′-5′-ADP.

The test described in FIG. 4 can be used as a diagnostic tool to distinguish non-2′-5′-ADP binding versus 2′-5′-ADP binding CPRs. This is important in the context of the development of inhibitors against CPRs as it allows one to examine different species, strains or individuals to determine if they have non-2′-5′-ADP binding CPR. Such enzymes present good pesticide targets since they differ to the 2′-5′-ADP binding human CPR counterpart¹⁹.

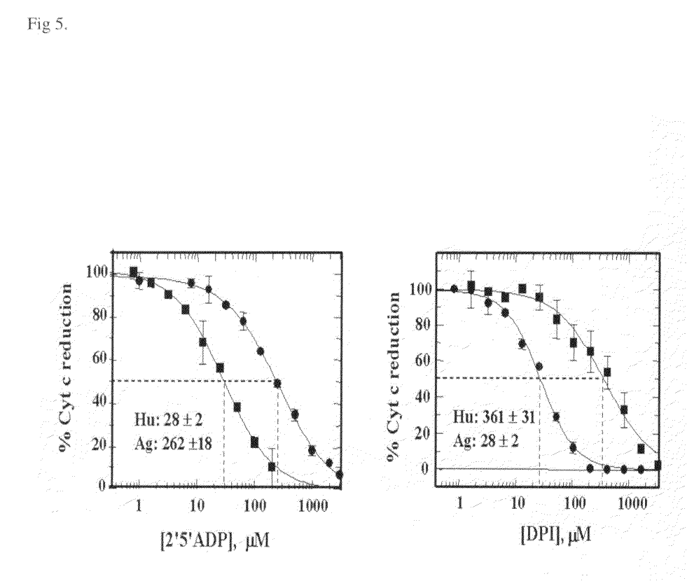

FIG. 5 shows inhibition of human and mosquito CPR by 2′ 5′ ADP and diphenyl iodonium (DPI). Micromolar IC₅₀ values for inhibition of cytochrome c reduction by human (squares) and mosquito (circles) are indicated in each graph on bottom left. Human CPR (IC_{5O}=28 μM) is ˜10 fold more sensitive to 2′ 5′-ADP inhibition (IC₅₀=262 μM), while mosquito CPR (IC₅₀=28 μM) is ˜10 fold more sensitive to DPI than human (IC₅₀=361 μM). Measurement of cytochrome c reduction was carried out at 25° C. with 50 μM cytochrome c and 0.75 pmol purified A. gambiae CPR or human CPR as described¹⁹, using different concentrations of 2′5′-ADP or DPI. A. gambiae and human CPR reactions were initiated by the addition of 30 μM or 15 μM NADPH respectively, corresponding to their apparent Kₘ values. Errors are deviation from the fit of the curve (GraFit 5.06).

FIG. 6 shows a SDS-polyacrylamide gel of purified A. gambiae CPR. Lane 1 shows the kilodalton molecular weight standards, with sizes indicated on left. Lane 2 shows a partially purified histidine tagged AgCPR, which has been eluted off a nickel affinity column with 300 mM imidazole. Lane 3 shows purified AgCPR that has been cleaved with thrombin to remove the N-terminal histidine tag.

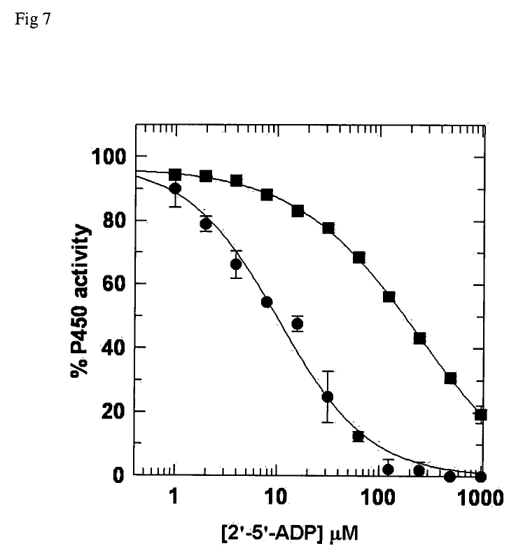

FIG. 7 shows inhibition of human and mosquito P450 activities by 2′-5′-ADP. Micromolar IC₅₀ values calculated for the inhibition of mosquito CYP6Z2 BR dealkylation (squares) and human CYP3A4 BQ oxidation (circles) are shown. Human P450 activity is approximately 20 fold more sensitive (IC₅₀=10±1 microM) than A. gambiae P450 (IC₅₀=234±28 microM). Error bars show standard deviation for two independent experiments. Measurement of P450 activity was carried out using E. coli membranes coexpressing A. gambiae CYP6Z2 and AgCPR or human CYP3A4 and human CPR. 7-benzyloxyresorufin (BR) and 7-benzyloxyquinoline (BQ) assays were performed in 200 μl reactions consisting of 50 mM potassium phosphate buffer, pH 7.4; 5 pmol CYP6Z2 or 20 pmol CYP3A4; 5 μM BR or 100 μM BQ, 125 microM NADPH and 0-1 mM 2′-5′-ADP. Rates were recorded for 5 min at 37° C. using a fluorescence plate reader (Labsystems Fluoroskan Ascent-FL) set to measure λ_{Ex 530} λ_{Em585} (BR assay) or λ_{Ex 405} λ_{Em530} (BQ assay). Percentage activities were calculated and IC₅₀ curves were plotted using GraFit version 5.

### MATERIALS & METHODS

### RNA Interference and Permethrin Resistance Assays

dscpr and dscpr* constructs were created by insertion of a 700 and 500 base pair XhoI restriction enzyme fragments of the cpr cDNA clone, respectively, into PLL10; a plasmid which carries two T7 polymerase primer sites in opposite orientation surrounding a multiple cloning site⁵. DsRNA was generated as described by Osta²⁰, following the standard protocol of the Ambion Megascript kit using a template consisting of 500 ng of KpnI digested pLLdscpr and an equal quantity of an EcoRI digestion of the same plasmid. RNAs were quantified spectrophotometrically, diluted to 3 μg/ml and analysed by ethithium bromide gel electrophoresis. RNAs synthesised from premixed, reverse complementary T7 polymerase templates spontaneously form dsRNA in vitro, which migrates slightly slower than the equivalent dsDNA and requires no additional annealing steps prior to use. Batches of 100 one to two day old female mosquitoes were divided into two groups and injected with 69 μl of either dscpr or dsgfp RNAs using a hand held Drummond nanoinjector II. Four days later, sub-groups of 19-20 mosquitoes were exposed to permethrin for 20 minutes using the standard WHO exposure kits (http://www.who.int/whopes/resistance/en/WHO_CDS_CPE_PVC_{—}2001.2.pdf) and impregnated papers (0.75% permethrin). 24 hours later, dead mosquitoes were counted. Three replicate biological experiments were performed.

### Immuno-Localization of cpr

Midgut and abdomen immuno-staining was performed essentially as described²¹. CPR was localized by sequential incubation with affinity purified CPR antisera (1/200) and appropriate fluorescent tag. Co-staining was performed with TO-PRO 3 (1/5000-Molecular Probes, DAPI, or Nuclear pore Mab414 (BabCo) and mouse anti-integrin antibodies with appropriate secondary antibodies as described in the figure legends, Localization of CPR in heads and their appendages was performed essentially as described²².

### Cloning, Expression and Purification of CPRs

Cloning of A. gambiae (Ag) CPR cDNA is described in Nikou et al 2003. Two nucleotide changes were found in the coding sequence relative to the published sequence²³; T689C and C1375T, producing Val230Ala and His459Tyr changes respectively. The membrane anchor sequence was deleted by removal of amino acids 2-63 by PCR, using PFU polymerase (Stratagene) and the following oligonucleotides; forward primer: CGCG GAT CCG ATG ACG ATG ACG ATG GTG GAG ACC and reverse primer: TTC GGA TCC TTA GCT CCA CAC GTC CGC CGA. (The BamHI sites are underlined and the start and stop codons are indicated in bold). The PCR product was digested with BamH I and subcloned into the expression vector pET-15b (Novagen). This expression vector has an in-frame 6× Histidine tag and thrombin cleavage sequence, which enables metal affinity purification and tag removal. This facilitates purification of the mosquito CPR which does not readily bind to 2′-5′-ADP sepharose, the usual affinity matrix for this enzyme class. Constructs were confirmed by DNA sequencing. AgCPR was expressed in E. coli strain BL21(DE3) pLysS and nickel-affinity purified as described previously for human CPR domains²⁴. The human CPR was purified as described by Dohr¹⁹. Protein purity was >95% as assessed by SDS-PAGE gel electrophoresis.

### Production of Antibodies.

Rabbit polyclonal antibodies to AgCPR were made by Moravian-Biotechnology Ltd, Brno, Czech Republic. Both antisera were affinity-purified against ˜100 μg purified recombinant CPR bound to nitrocellulose using ImmunoPure Gentle Ag/Ab Binding and Elution buffers (Pierce, Rockford, Ill., U.S.A.) according to manufacturers instructions.

### Cytochrome c and NADPH Kinetics

Cytochrome c assays and enzyme kinetic measurements were carried out 25° C. as described¹⁹, using 0.75 pmol purified AgCPR or hCPR. Apparent kinetic parameters (non-linear fitting: Michaelis-Menten equation) and IC₅₀ values were calculated using GraFit version 5.06.

### Comparison of Mosquito and Fruit Fly 2′-5′-ADP Binding Characteristics.

0.1 g of frozen adult A. gambiae or D. melanogaster flies were added to a pre-chilled mortar and ground to a powder in the presence of liquid nitrogen. The powder was transferred to a 50 ml Falcon tube and 2.5 mls of cold solubilisation buffer (20 mM Tris-Cl pH 8; 10% glycerol; 100 mM KCl and 20 mM CHAPS) added. The suspensions were mixed on a rotary wheel at 4° C. for 2 hrs to solubilise the membrane bound proteins, then centrifuged (5 min; 12,000 rpm; 4° C.) to clarify. 200 μl of 2′ 5′ ADP sepharose was loaded into a mini-column (a 1 ml Gillson pipette tip, plugged with glass-wool) and equilibriated with 5 ml of solubilisation buffer. The clarified whole fly protein extracts were applied to the column and the flow-through collected. The column was washed with 5 ml of solubilisation buffer to remove non-binding proteins. To elute the bound proteins, 3×200 μl of 50 mM 2′ AMP in 0.5×solubilisation buffer was added to the mini-columns, and the 3×200 μl elution fractions collected in Eppendorf tubes. To detect CPR, 25 μl each of solubilised pre-column extract (total fly protein); flow through (unbound protein) and eluted proteins were run on a NuPAGE 4-12% Bis-Tris Gel (Invitrogen; UK) and transferred onto Protran® nitrocellulose membrane (Schleicher & Schuell; Germany) according to the manufacturer's instructions. The membrane was blocked overnight at 4° C. in TBST & 5% milk powder, then incubated for 1 hr with a 1:10000 dilution of anti-Ag CPR primary antibody. After washing (3×10 min in TBST) the membrane was incubated for 1 hr with 1:3000 dilution of HRP anti-rabbit IgG (SAPU; UK). The membranes were washed as before and the proteins were visualised by chemiluminescence using the ECL (Amersham Biosciences) kit according to the manufacturers instructions. Functional expression of CYP6Z2-CYP6Z2 cDNAs was isolated as described²³. For E. coli expression, CYP6Z2 cDNA was fused to a bacterial OmpA leader sequence as previously described (Pritchard, M. P., et al. (1998) Pharmacogenetics 8, 3342; Pritchard et al (1.997) Arch Biochem Biophys 345, 342-354) by PCR with OmpA forward primer: G GAA TTC CAT ATG AAA AAG ACA GCT AT (Nde I site underlined, initiation codon in bold); OmpA/CYP6Z2 fusion primer (reverse orientation): GAG CAC GAG AAA GAT CAC GGC CGC AAC AAG AGC AAG AGT ATA AAC AGC CAT CGG AGC GGC CTG CTG CGC TAC GGT AGC GAA; CYP6Z2 reverse primer: CGG GAA TTC TCA CTT TCT ATG GTC TAT CCT CAT (EcoR I site underlined, stop codon in bold). The PCR fragment was digested with Nde I/EcoR I and ligated into pB13 (modified pCW vector). For functional expression CYP6Z2 was co-expressed with full-length A. gambiae CPR cloned into a compatible pACYC vector. A. gambiae CPR cDNA was fused by PCR to a PelB leader sequence (Forward primer: C GGG ATC CAT ATG AAA TAC CTG CTG CCG ACC GCT GCT GCT GCT CTG CTG CTC CTC GCT GCC CAG CCG GCG ATG GCC ATG GAC GCC CAG ACA GAA ACG GAA GTG (BamH I site underlined and start codon in bold) and reverse primer: CCG GAA TTC TTA GCT CCA CAC GTC CGC CGA GTA TCG TTT (EcoR I site underlined and stop codon in bold). The PCR product was digested with BamH I/EcoR I and cloned into pB13. A cassette containing the Ptac-Ptac promotor from pB13 and PelB AgCPR was excised using an EcoR V/Bgl II digest, and cloned into EcoR V/BamH I digested pACYC 184 (New England Biolabs), disrupting the tetracycline resistance gene (pACYC-AgCPR). Competent E. coli JM109 cells were co-transformed with OmpA 6Z2 & pACYC-AgCPR, and expression, membrane isolation and determination of P450 and CPR content was carried out as previously described (Pritchard, M. P., et al. (1998) Pharmacogenetics 8, 33-42).

For P450 assays, E. coli membranes co-expressing human CYP3A4 and CPR were produced as described. 7-benzyloxyresorufin (BR) and 7-benzyloxyquinoline (BQ) assays were performed in 96 well white plates in a total volume of 200 μl consisting of 50 mM potassium phosphate buffer, pH 7.4; 5 pmol CYP6Z2 or 20 pmol CYP3A4; 5 μM BR or 100 μM BQ, with 0-1 mM 2′-5′-ADP. Plates were preincubated for 5 min at 37° C. before addition of 5 μl of 5 mM NADPH. Rates were recorded for 5 min at 37° C. using a fluorescence plate reader (Labsystems Fluoroskan Ascent-FL) set to measure λ_{Ex 530} λ_{Em585} (BR assay) or λ_{Ex 405} λ_{Em530} (BQ assay).

Percentage activities were calculated and IC₅₀ curves were plotted using GraFit version 5.

### EXAMPLE 1

### CPR is Highly Abundant in Oenocytes

The tissue distribution of the P450 complex has been poorly described in mosquitoes. Using an affinity purified antibody against CPR, a peptide of the expected size was detected in A. gambiae extracts derived from dissected head (with appendages), gut (with Malphigian tubules), and the abdomen wall. CPR was only weakly detected in the thorax, which is largely comprised of muscle, fat body and salivary glands (not shown).

The same antibody revealed that the cellular distribution of CPR can be divided into three main tissues; antenna, midgut epithelia and oenocytes. In the head, staining was prominent in a subset of cells within the antennae (FIG. 2F), which is consistent with reported high levels of CPR in this organ in Drosophila melanogaster²⁵. It has been proposed to be involved in the metabolism of chemical odorants, clearing the olfactory organ from accumulating chemicals. CPR was also observed in midgut epithelial cells (FIGS. 2A-C), and was highly expressed in the foregut epithelium. P450 dependant metabolism within these tissues is probably important to neutralise toxins acquired during feeding on plant material² and possibly in the blood meal²⁶. The principal (type 1) cells of the Malphigian tubules were also specifically labelled (FIG. 2G). These cells are involved in maintaining ion homeostasis and are known sites of P450 dependant ecdysone metabolism in Drosophila and other insects^{27,28}.

Very intensely stained large cells (>25 μm) on the abdomen wall of adults were observed (FIGS. 2D,E). These cells were found in distinct subcuticular clumps that form rows on each abdominal segment (FIG. 2D), predominantly on the ventral half of the abdomen wall. They clearly overlap the brown cuticle pigmentation in the overlay of brightfield and fluorescence images (FIG. 2D) and may thus be involved in its formation. Based on anatomical description and comparison with other insects, particularly Drosophila²⁹, these cells are identified as oenocytes.

Oenocytes are a major focus of Drosophila developmental research as their identity is controlled by a single homeotic gene (Abdominal A)³⁰, however, their functional role is still unresolved. Based on gene products expressed, these cells have been ascribed numerous endocrine and secretory functions including the regulation of ecdysteroids and production of pheromones³¹, glycogen storage³², and hydrocarbon/lipid synthesis³³. It is probable that CPR is associated with several of these metabolic activities, many of which have been linked to P450 activities. In Drosophila, oenocytes are thought to be the major site of heme biosynthesis³⁴, suggesting a role in cytochrome P450 production. In adult A. gambiae the oenocytes are restricted to the ventral body wall, which contrasts with Drosophila in which these cells are evenly distributed on both abdominal surfaces. This may reflect different physiological requirements in the two species.

Overall, the tissue distribution in mosquito suggests key physiological roles for CPR in metabolic processes and pheromone production/metabolism, consistent with known or expected functional involvement of insect P450s². In addition, the abundant presence of an archetypal detoxification enzyme in the oenocytes suggests that this cell type has some functional equivalence to hepatocytes.

### EXAMPLE 2

### CPR Knockdown in Oenocytes Correlates with Increased Permethrin Susceptibility

To facilitate CPR silencing, dsRNAs corresponding either to the CPR gene (dsCPR) or to the control green fluorescent protein, gene (dsGFP) were injected into 1-2 day old adults. These were allowed to recover for 4 days. No significant difference in survival was noted between experimental and control samples following injection and recovery (not shown). Western analysis of extracts taken from abdomen, gut and heads dissected from dsGFP and dsCPR treated mosquitoes indicted that CPR depletion was most efficient in the abdomen (−90%), with a smaller reduction evident in midgut extracts (−50%) and negligible differences in the head extracts (FIG. 3a). In whole mount abdomens, we also noted a strong reduction in CPR staining in oenocytes (FIG. 3b). We have thus established that CPR expression can be effectively knocked down in the abdomen, particularly in the oenocytes, allowing us to examine their role in insecticide metabolism in vivo.

The present inventors investigated the response to permethrin, a pyrethroid insecticide currently used in malaria control programmes¹. dsRNA-treated mosquitoes were challenged with a fixed permethrin dose at different exposure times and their survival monitored 24 hours later, according to the WHO guidelines e.g. http://www.who.int/whopes/resistance/en/WHO_CDS_CPE_PVC_{—}2001.2.pdf. Initial experiments defined an appropriate exposure in the laboratory strain we used. At a level at which the dsGFP controls showed approximately 40% lethality, the dsCPR treated mosquitoes showed a 2 fold increase in susceptibility to permethrin (˜80%). This finding is reminiscent to the severely compromised ability of mice carrying a conditional deletion of hepatic CPR⁴ to metabolise drugs such as pentobarbital or the analgesic acetaminophen⁴. Our results indicate a key physiological role for CPR in protection against permethrin in the mosquito, presumably through P450 metabolism. They also suggest CPR as a viable target for the development of inhibitors to enhance and prolong the effectiveness of permethrin and potentially other insecticides metabolised by the P450 enzyme complex.

### EXAMPLE 3

### Mosquito CPR Exhibits Distinctive Biochemical Differences in Relation to Fruit Fly and Human CPR

To further characterise A. gambiae CPR the present inventors compared the biochemical properties of the mosquito, fruit-fly and human enzymes. Since the A. gambiae CPR contains an NADPH binding domain²³, it night be expected to be purified easily from whole fly extracts through affinity purification using 2′-5′-ADP Sepharose (2′-5′-ADP Sepharose interacts strongly with NADP+-dependent dehydrogenases and other enzymes which have affinity for NADP+ (Amersham-Pharmacia Biotech 1999 handbook on Affinity Chromatography: Principles and Methods, edition AB). However, comparison of the purification of crude 2′-5′-ADP binding proteins from crude extracts of A. gambiae and the closely related dipteran species D. melanogaster show that the mosquito CPR does not bind 2′-5′-ADP (FIG. 4) and is thus clearly different with respect to the molecular recognition of adenosine. Soluble forms (i.e. lacking the N-terminal membrane anchor)¹⁹ of the catalytic regions of human and A. gambiae CPR were expressed in E. coli¹⁹. The soluble histidine tagged form of A. gambiae CPR purified over nickel agarose (FIG. 6) also failed to bind to 2′-5′-ADP resin, indicating that the lack of binding was not artefactual.

The relative enzymatic activities of human and A. gambiae CPR were measured through the reduction of cytochrome c (a surrogate electron acceptor used for measuring diflavin reductase activity³). These revealed functional similarities with human CPR as well as significant and unexpected biochemical differences. The binding affinities for cytochrome c were similar with Kₘ^{cytc} values for human and mosquito enzymes of 19 μM and 23 μM respectively. Rates of cytochrome c reduction were also alike, characterized by k_{cat} values of 3023 and 3099 nmoles cytochrome c reduced/nmole CPR/min⁻¹. A two-fold decrease in affinity for NADPH relative to the human protein was noted (Kₘ^{NADPH} 30 μM and 14 μM respectively). Overall, these experiments show comparable steady state rates of electron transfer from NADPH to cytochrome c.

An unexpected difference, however, was observed in the binding affinity for adenosine molecules (FIG. 4). NADPH is comprised of nicotinamide and adenosine-ribose moieties (i.e 2′,5′-ADP), which are proposed to bind in a bipartite mode to separate binding pockets of CPR¹⁹. A. gambiae CPR failed to bind to 2′,5′-ADP Sepharose, a standard affinity matrix used for purifying CPRs and related NADPH binding enzymes. We therefore compared the inhibitory effects of the 2′,5′-ADP fragment and found a ten-fold higher IC₅₀ for A. gambiae CPR (IC₅₀ 262 μM) in comparison to human CPR (IC₅₀ 28 μM) (FIG. 5). The structural reasons are still unclear, but may be associated with interactions involving 2′-phosphate, which is the major contributor to the high affinity binding of NADPH to CPR³⁵. Importantly, we also found that A. gambiae CPR was an order of magnitude more sensitive than human CPR to diphenyliodonium chloride (DPI) (FIG. 5), a widely used inhibitor of flavin-containing enzymes³⁶. These results highlight significant differences in binding interactions with small molecules that may be exploited to develop a mosquito CPR specific inhibitor.

### EXAMPLE 4

### Mosquito CPR Exhibits Distinctive Biochemical Differences in Relation to Human CPR in P450 Coupling

To confirm whether such differences in 2′-5′-ADP binding could be detected with physiological redox partners, we performed inhibition assays by co-expressing full length mosquito and human CPRs in E. coli membranes with one of their respective P450 partners, CYP6Z2²³ and CYP3A4 (Pritchard, M. P. et al (1997) Arch Biochem Biophys 345, 342-354). Measurements were made of the oxidation of the fluorogenic substrates 7-benzyloxyresorufin (BR) and 7-benzyloxyquinoline (BQ), which are metabolised by CYP6Z2 and CYP3A4 respectively (FIG. 7). Consistent with inhibition of cytochrome c reduction, the IC₅₀ of 2′,5′-ADP for the mosquito CYP6Z2 catalysed BR O-dealkylation was 234 μM, which was approximately twenty-fold greater than the IC₅₀ (10 μM) for human CYP3A4 BQ oxidation.

Furthermore, reciprocal pairings of mosquito and human CPRs and P450s were also examined (i.e. CYP6Z2 was co-expressed with human CPR and CYP3A4 with A. gambiae CPR), but we were unable to detect significant P450 activity with either cross pairing. In view of the high sequence homology of human and mosquito CPRs (40% identical and 60% similar residues)²³ and the fact that CPR homologues are generally interchangeable (Feyereisen, R. (1999) Annu Rev Entomol 44, 507-533), the failure of A. gambiae CPR to couple with human CYP3A4 suggests possible differences in surface charge or hydrophobicity affecting CPR-P450 contact, or interactions with the membrane.

In conclusion, this work identifies oenocytes as being one of the primary sites for CPR expression and by association P450 metabolism. This is strongly supported by the increase in susceptibility to permethrin following the depletion of CPR gene expression in oenocytes. Our results firmly establish the critical role of P450 monoxygenase complex in insecticide metabolism. They also identify CPR as a viable target for the development of functional inhibitors for use as insecticides. There are two compelling reasons to investigate this further. Firstly, CPR is encoded by a single gene that has a house-keeping role in furnishing electrons to all microsomal P450s. Its inhibition is therefore likely to be lethal to early development stages. Secondly, the low affinity of A. gambiae CPR for 2′,5′-ADP distinguishes it from all other CPRs that have been purified³⁷, including those from other insects. Although this initial findings merits further investigation it encourages die efforts to design or screen for selective inhibitory molecules that will specifically target A. gambiae.

### REFERENCES

1. Ranson, H. et al. Evolution of supergene families associated with insecticide resistance. Science 298, 179-81 (2002).
2. Feyereisen, R. Insect P450 enzymes. Annu Rev Entomol 44, 507-33 (1999).
3. Paine, M. J. I., Scrutton, N. S., Munro, A. W., Roberts, G. C. K. & Wolf, C. R. in Cytochromes P450: Structure, Mechanism and Biochemistry (ed. Ortiz de Montellano, P. R.) 115-148 (Kluwer Academic/Plenum Publishers, New York, 2004).
4. Henderson, C. J. et al. Inactivation of the hepatic cytochrome P450 system by conditional deletion of hepatic cytochrome P450 reductase. J. Biol. Chem. 278, 13480-13486 (2003).
5. Blandin, S. et al. Reverse genetics in the mosquito Anopheles gambiae: targeted disruption of the Defensin gene. EMBO Rep 3, 852-6 (2002).
6. Montgomery, M. K. RNA interference: historical overview and significance. Methods Mol Biol 265, 3-21 (2004).
7. Mello, C. C. & Conte, D., Jr. Revealing the world of RNA interference. Nature 431, 338-42 (2004).
8. Sanchez-Vargas, I. et al. RNA interference, arthropod-bone viruses, and mosquitoes. Virus Res 102, 65-74 (2004).
9. Andreadis, T. G., Becnel, J. J. &; White, S. E. Infectivity and pathogenicity of a novel baculovirus, CuniNPV from Culex nigripalpus (Diptera: Culicidae) for thirteen species and four genera of mosquitoes. J Med Entomol 40, 512-7 (2003).
10. Becnel, J. et al. Epizootiology and transmission of a newly discovered baculovirus from the mosquitoes Culex nigripalpus and C. quinquefasciatus. J Gen Virol 82, 275-82 (2001).
11. Shapiro, A. M., Becnel, J. J. & White, S. E. A nucleopolyhedrovirus from Uranotaenia sapphirina (Diptera: Culicidae). J Invertebr Pathol 86, 96-103 (2004).
12. Travanty, E. A. et al. Using RNA interference to develop dengue virus resistance in genetically modified Aedes aegypti. Insect Biochem Mol Biol 34, 607-13 (2004).
13. Kohl, A., Billecocq, A., Prehaud, C., Yadani, F. Z. & Bouloy, M. Transient gene expression in mammalian and mosquito cells using a recombinant Semliki Forest virus expressing T7 RNA polymerase. Appl Microbiol Biotechnol 53, 51-6 (1999).
14. Sambrook, J. & Russell, D. W. Molecular Cloning: A Laboratory Manual (ed. J., S.) (Cold Spring Harbour Laboratory Press, New York, 2001).
15. Vermilion, J. L. & Coon, M. J. Purified liver microsomal NADPH-cytochrome P-450 reductase. Spectral characterization of oxidation-reduction states. J Biol Chem 253, 2694-704. (1978).
16. Pritchard, M. P. et al. Functional co-expression of CYP2D6 and human NADPH-cytochrome P450 reductase in Escherichia coli. Pharmacogenetics 8, 33-42. (1998).
17. Paine, M. J., Gilham, D., Roberts, G. C. & Wolf, C. R. Functional high level expression of cytochrome P450 CYP2D6 using baculoviral expression systems. Arch Biochem Biophys 328, 143-50. (1996).
18, Wang, M. et al. Three-dimensional structure of NADPH-cytochrome P450 reductase: prototype for FMN- and FAD-containing enzymes. Proc Natl Acad Sci USA 94, 841.1-6. (1997).
19. Dohr, O., Paine, M. J., Friedberg, T., Roberts, G. C. & Wolf, C. R. Engineering of a functional human NADH-dependent cytochrome P450 system. Proc Natl Acad Sci USA 98, 81-6. (2001).
20. Osta, M. A., Christophides, G. K. & Kafatos, F. C. Effects of mosquito genes on Plasmodium development. Science 303, 2030-2 (2004).
21. Han, Y. S., Thompson, J., Kafatos, F. C. & Barillas-Mury, C. Molecular interactions between Anopheles stephensi midgut cells and Plasmodium berghei: the time bomb theory of ookinete invasion of mosquitoes. Embo J 19, 6030-40 (2000).
22. Pitts, R. J., Fox, A. N. & Zwiebel, L. J. A highly conserved candidate chemoreceptor expressed in both olfactory and gustatory tissues in the malaria vector Anopheles gambiae. Proc Natl Acad Sci USA 101, 5058-63 (2004).
23. Nikou, D., Ranson, H. & Hemingway, J. An adult-specific CYP6 P450 gene is overexpressed in a pyrethroid-resistant strain of the malaria vector, Anopheles gambiae. Gene 318, 91-102 (2003).
24. Smith, G. C. M., Tew, D. G., and Wolf, C. R. Dissection of NADPH-cytochrome P450 oxidoreductase into distinct functional domains. Proc Natl Acad Sci USA 91, 8710-8714 (1994).
25. Hovemann, B. T., Sehlmeyer, F. & Malz, J. Drosophila melanogaster NADPH-cytochrome P450 oxidoreductase: pronounced expression in antennae may be related to odorant clearance, Gene 189, 213-9 (1997).
26. Luckhart, S. et al. Mammalian transforming growth factor beta1 activated after ingestion by Anopheles stephensi modulates mosquito immunity. Infect Immun 71, 3000-9 (2003).
27. Winter, J., Bilbe, G., Richener, H., Sebringer, B. & Kayser, H. Cloning of a cDNA encoding a novel cytochrome P450 from the insect Locusta migratoria: CYP6H1, a putative ecdysone 20-hydroxylase. Biochem Biophys Res Commun 259, 305-10 (1999).
28. Petryk A et al. Shade is the Drosophila P450 enzyme that mediates the hydroxylation of ecdysone to the steroid insect molting hormone 20-hydroxyecdysone. Proceedings of the National Academy of Science USA 100, 13773-8 (2003).
29. Miller, A. in Biology of Drosophila (ed. Demerec, M.) 420-534 (Haner, N.Y., 1950).
30. Brodu, V., Elstob, P. R. & Gould, A. P. abdominal A specifies one cell type in Drosophila by regulating one principal target gene. Development 129, 2957-63 (2002).
31. Ferveur, J. F. et al. Genetic feminization of pheromones and its behavioral consequences in Drosophila males. Science 276, 1555-8 (1997).
32. Pennisi E. Old Flies May Hold Secrets of Aging. Science 290, 2048 (2000).
33. Fan, Y., Zurek, L., Dykstra, M. J. & Schal, C. Hydrocarbon synthesis by enzymatically dissociated oenocytes of the abdominal integument of the German Cockroach, Blattella germanica. Naturwissenschaften 90, 121-6 (2003).
34. Ruiz de Mena, I., Fernandez-Moreno, M. A., Bornstein, B., Kaguni, L. S. & Garesse, R. Structure and regulated expression of the delta-aminolevulinate synthase gene from Drosophila melanogaster. J. Biol. Chem. 274, 37321-8 (1999).
35. Murataliev, M. B. & Feyereisen, R. Interaction of NADP(H) with oxidized and reduced P450 reductase during catalysis. Studies with nucleotide analogues. Biochemistry 39, 5066-74. (2000).
36. Tew, D. G. Inhibition of cytochrome P450 reductase by the diphenyliodonium cation. Kinetic analysis and covalent modifications. Biochemistry 32, 10209-15 (1993).
37. Murataliev, M. B., Arino, A., Guzov, V. M. & Feyereisen, R. Kinetic mechanism of cytochrome P450 reductase from the house fly (Musca domestica). Insect Biochem Mol Biol 29, 233-42 (1999).

1. A method of pest treatment comprising administering an agent to said pest, which agent is effective in reducing activity and/or expression of said pest's cytochrome P450 reductase.

2. The method according to claim 1 wherein a pesticide is to be administered in combination with or concurrently with the agent to said pest.

3. The method according to claim 1 wherein reducing said expression is achieved by use of antisense oligonucleotides designed against the CPR gene and/or promoter sequences, or by the use of RNAi.

4. A method of killing pests, especially mosquitoes comprising administering a double-stranded RNA molecule corresponding to at least a portion of the gene sequence of the pest's CPR gene, for inhibiting expression of the mosquito's CPR gene, by the process of RNAi, and/or a chemical agent capable of reducing activity of mosquito CPR optionally in combination with a pyrethroid pesticide, such as permethrin.

5. A pesticide formulation for use in killing pests, the formulation comprising a dsRNA molecule corresponding to at least a portion of the gene sequence of the pest's CPR gene and/or a chemical agent capable of reducing activity of the pest's CPR, optionally in combination with a pyrethroid pesticide, such as permethrin.

6. A pesticide formulation, the formulation comprising a genetically engineered insect virus which comprises an inserted nucleic acid encoding at least a portion of a pest's CPR gene and wherein said nucleic acid is capable of being expressed as a dsRNA molecule and optionally a pyrethroid pesticide, such as permethrin.

7. The formulation according to claim 6, wherein the genetically engineered insect virus is a baculovirus that expresses said dsRNA molecule in pest cells infected with the recombinant baculovirus.

8. Use of a genetically engineered insect virus capable of expressing a dsRNA molecule encoded by a portion of a pest's CPR gene for the manufacture of a pesticide to be administered, optionally in combination with a chemical pesticide, to treat pests such as insects.

9. The method or use according to claim 6 wherein the genetically engineered virus is a polyhedrosis virus.

10. The method or use according to claim 9, wherein the polyhedrosis virus is Lymantria dispar NPV (gypsy moth NPV), Autographa californica MNPV, Anagrapha falcifera NPV (celery looper NPV), Spodoptera litturalis NPV, Spodoptera frugiperda NPV, Heliothis armigera NPV, Mamestra brassicae NPV, Choristoneura fumiferana NPV, Trichoplusia ni NPV, Heliocoverpa zea NPV, and Rachiplusia ou NPV.

11. The method or use according to claim 6 wherein the genetically engineered virus is a CuniNPV, UrsaNPV, Recombinant Sindbis virus or a Semliki Forest virus (SFV) expressing T7 RNA polymerase (T7-RP).

12. The method, or formulation, or use according to claim 2 wherein the pesticide is a Na.sup+channel agonists (i.e. pyrethroids), Na.sup+channel blocking agents (i.e. pyrazolines), acetylcholinesterase inhibitors (i.e. organophosphates and carbamates), nicotinic acetylcholine binding agents (e.g. imidacloprid), gabaergic binding agents (e.g. emamectin and fipronil), octapamine agonists or antagonists (i.e. formamidines), and oxphos uncouplers (e.g. pyrrole insecticides).

13. The method, formulation or use according to claim 2 wherein the pesticide is applied by means such as spraying, atomising, dusting, scattering or pouring and may be formulated for such applications as powders, dusts, granulates, as well as encapsulations such as in polymer substances.

14. The method, formulation or use according to claim 2 wherein the pesticide and dsRNA/recombinant baculovirus will be admixed in desired proportions, and may typically include inert carriers such as clay, lactose, defatted soy bean powder, and the like to assist in application.

15. A method of screening for a potential pesticide comprising the steps of:
a) providing a pest cytochrome P450 reductase (CPR) model system comprising CPR from a pest organism and a substrate capable of being reduced by said CPR;
b) contacting a test pesticide agent with said system;
c) initiating reduction of said substrate by the addition of an electron donor to said system; and
d) observing any change in a rate of substrate reduction in comparison to a rate of substrate reduction in the absence of said test insecticide agent.

16. The method, formulation or use according to claim 1 wherein the pest is selected from Dictyoptera (cockroaches); Isoptera (termites); Orthoptera (locusts, grasshoppers and crickets); Diptera (house flies, mosquito, tsetse fly, crane-flies and fruit flies); Hymenoptera (ants, wasps, bees, saw-flies, ichneumon flies and gall-wasps); Anoplura (biting and sucking lice); Siphonaptera (fleas); and Hemiptera (bugs and aphids), as well as arachnids such as Acari (ticks and mites) and insect bourne protozoan parasites (Trypanosoma, Leishmania, Giardia, Trichomonas, Entamoeba, Naegleria, Acanthamoeba, Plasmodium, Toxoplasma, Cryptosporidium, Isospora and Balantium)

17. The method, formulation or use according to claim 16 wherein the pest is the mosquito.

18. A computer system, intended to generate structures and/or perform rational drug design for Anopheles sp. P450 reductase, or homologues or mutants, the system containing either (a) atomic coordinate data, said data defining the three-dimensional structure of Anopheles sp. P450 reductase, or at least selected coordinates thereof; (b) structure factor data of Anopheles sp. P450 reductase recorded thereon, the structure factor data being derivable from the atomic coordinate data or (c) a Fourier transform of atomic coordinate data or at least selected coordinates thereof.

19. Computer readable media with either (a) atomic coordinate data recorded thereon, said data defining the three-dimensional structure of Anopheles sp. P450 reductase, or at least selected coordinates thereof; (b) structure factor data for Anopheles sp. P450 reductase recorded thereon, the structure factor data being derivable from the atomic coordinate data or (c) a Fourier transform of said atomic coordinate data, or at least selected coordinates thereof.

20. A method for modelling the interaction between Anopheles sp. P450 reductase and an agent compound which modulates said reductase activity, comprising the steps of:
(a) employing three-dimensional atomic coordinate data to characterise the Anopheles sp. P450 reductase binding site; (b) providing the structure of said agent compound; and (c) fitting said agent compound to the binding site.

21. A method for identifying an agent compound (e.g. an inhibitor) which modulates Anopheles sp. P450 reductase activity, comprising the steps:
(a) employing three-dimensional atomic coordinate data according to characterise at least one Anopheles sp. P450 reductase binding site; (b) providing the structure of a candidate agent compound; (c) fitting the candidate agent compound to the binding site(s); and (d) selecting the candidate agent compound.

22. The system, media or method according to claim 1 wherein the Anopheles sp. is Anopheles gambiae.

23. A method of determining whether or not a pest is likely to be susceptible to a pesticide identified according to the present invention, comprising the steps of:
a) obtaining said pest;
b) homogenising said pest, so as to release said pest's cytochrome P450 reductase (CPR);
c) admixing said homogenate containing CPR with ADP sepharose—if necessary removing ADP sepharose binding contaminants from the homogenate first before the ADP-affinity binding step; and
d) detecting whether or not said CPR substantially binds to ADP sepharose.


SEQUENCE LISTING (7 sequences)

SEQ ID NO: 1 - DNA, 34 bp, Artificial Sequence
  Primer
cgcggatccgatgacgatgacgatggtggagacc

SEQ ID NO: 2 - DNA, 30 bp, Artificial sequence
  Primer
ttcggatccttagctccacacgtccgccga

SEQ ID NO: 3 - DNA, 27 bp, Artificial sequence
  Primer
ggaattccatatgaaaaagacagctat

SEQ ID NO: 4 - DNA, 81 bp, Artificial sequence
  Primer
gagcacgagaaagatcacggccgcaacaagagcaagagtataaacagccatcggagcggcctgctgcgctacggtagcgaa

SEQ ID NO: 5 - DNA, 33 bp, Artificial sequence
  Primer
cgggaattctcactttctatggtctatcctcat

SEQ ID NO: 6 - DNA, 103 bp, Artificial sequence
  Primer
cgggatccatatgaaatacctgctgccgaccgctgctgctgctctgctgctcctcgctgcccagccggcgatggccatggacgcccagacagaaacggaagtg

SEQ ID NO: 7 - DNA, 39 bp, Artificial sequence
  primer
ccggaattcttagctccacacgtccgccgagtatcgttt